(19) United States
(12) Patent Application Publication
(10) Pub. No.: US 2024/0069014 A1
(43) Pub. Date: Feb. 29, 2024

(54) SARS-CoV-2 Rapid Detection Device

(71) Applicant: City University of Hong Kong, Hong Kong (CN)

(72) Inventors: Xinge YU, Hong Kong (CN); Hu LI, Hong Kong (CN); Jingkun ZHOU, Hong Kong (CN)

(21) Appl. No.: 17/821,506

(22) Filed: Aug. 23, 2022

(51) Int. Cl. G01N 33/543; A61B 10/00; G01K 7/22; G01N 27/12; G01N 33/569

(52) U.S. Cl. CPC G01N 33/5438; A61B 10/00; G01K 7/22; G01N 27/125; G01N 33/56983; A61B 2010/0087

(57) ABSTRACT

An integrated device for rapid detection of SARS-CoV-2 is disclosed. The integrated device includes a front-end system, a back-end system, and a microfluidic channel. The front-end system includes a detection region and a control region. The detection region and the control region have similar structures, each including an interdigital electrode and a graphene film. The microfluidic channel is arranged to cover the detection region so as to allow the interdigital electrode of the detection region to be exposed to the breath sample. The graphene film of the detection region has a surface resistance higher than that of the graphene film of the control region when the detection region is exposed to a selected virus. The back-end system is configured to detect and compare the surface resistance of the graphene films of the detection region and the control region for determining whether the selected virus is present in the breath sample.

### FIELD OF THE INVENTION

The present invention generally relates to a device for fast diagnosis of COVID-19, and particularly relates to a flexible, multifunctional device capable of communicating wirelessly for transmitting information pertaining to the medical condition to the user.

### BACKGROUND OF THE INVENTION

Severe acute respiratory syndrome coronavirus 2 (SARS-CoV-2) is a virus recently identified as the cause of an outbreak of the Coronavirus disease 2019 (COVID-19) with an increasing number of patients with severe symptoms and deaths. The virus is highly contagious when the patient is infected, both when symptoms are developed and when there are no or very minor symptoms. To reduce the transmission of SARS-CoV-2 and to safeguard public health, it is crucial to detect an infection as early as possible with a sensitive, reliable test without the need for laboratory equipment.

Currently available Coronavirus diagnostic tests can be categorized into antigen tests and polymerase chain reaction (PCR) based nucleic acid tests. The PCR-based tests require extensive laboratory infrastructure and take a longer time to get results. The antigen tests can give a result sooner, which offers simple, cost-effective, portable, and easy to use methods, particularly for screening. However, the disadvantage of the antigen tests is the lower accuracy for the infected patient in an early stage prior to showing symptoms.

Accordingly, there is a need in the art for a device that can perform SARS-CoV-2 rapid detection on a patient with and without symptoms. Furthermore, other desirable features and characteristics will become apparent from the subsequent detailed description and the appended claims, taken in conjunction with the accompanying drawings and this background of the disclosure.

### SUMMARY OF THE INVENTION

In accordance with an embodiment of the present disclosure, an integrated device for analyzing a breath sample and rapid detection of SARS-CoV-2 is disclosed. The integrated device includes a front-end system, a back-end system, and a microfluidic channel. The front-end system includes a detection region and a control region. The detection region and the control region have similar structures, each including an interdigital electrode and a graphene film. The microfluidic channel is arranged to cover the detection region so as to allow the interdigital electrode of the detection region to be exposed to the breath sample. The graphene film of the detection region has a surface resistance higher than that of the graphene film of the control region when the detection region is exposed to a selected virus. The back-end system is configured to detect and compare the surface resistance of the graphene films of the detection region and the control region for determining whether the selected virus is present in the breath sample.

In accordance with a further embodiment of the present disclosure, the microfluidic channel comprises a plurality of gas channels and a trapping chamber holding pre-injected Phosphate-buffered saline (PBS) for defining a gas-liquid interface. The graphene film and the interdigital electrode of the detection region are placed inside the trapping chamber and immersed in the pre-injected PBS. The breath sample passes through the gas-liquid interface for capturing the selected virus in the pre-injected PBS.

In accordance with a further embodiment of the present disclosure, the pre-injected PBS is injected with a molecular linker and a spike-binding antibody, wherein the spike-binding antibody is arranged to capture the spike protein of SARS-CoV-2, and the surface resistance of the graphene film of the detection region is increased when exposed to the selected virus with the SARS-CoV-2 linked to the graphene film by the molecular linker.

In accordance with a further embodiment of the present disclosure, the spike-binding antibody is an anti-Coronavirus spike neutralizing antibody (40592-MM45) for recognizing a Delta variant, or an anti-Coronavirus spike neutralizing antibody (40591-MM48) for recognizing an Omicron variant.

In accordance with a further embodiment of the present disclosure, the molecular linker comprises cross-linking collagen polymers using 1-ethyl-3-(3-dimethylaminopropyl)carbodiimide (EDC) and N-hydroxysuccinimide (NHS).

In accordance with a further embodiment of the present disclosure, a first end of the EDC/NHS combines with the graphene film, and a second end of the EDC/NHS combines with the spike-binding antibody.

In accordance with a further embodiment of the present disclosure, the plurality of gas channels comprise plural gas inlets and a gas outlet, which are arranged outside and around the trapping chamber, wherein the gas outlet is positioned proximate to a gas side of the gas-liquid interface, and the plural gas inlets are positioned proximate to a liquid side of the gas-liquid interface.

In accordance with a further embodiment of the present disclosure, the interdigital electrode is covered by the graphene film and adhesively attached to an acrylic sheet.

In accordance with a further embodiment of the present disclosure, the back-end system comprises a thermistor or other temperature sensors for detecting a breath temperature of the breath sample, wherein the breath temperature is mapped to a temperature compensation curve for estimating a forehead temperature.

In accordance with a further embodiment of the present disclosure, the back-end system is further configured to monitor a variation in the surface resistance of the graphene film of the control region for determining a respiratory rate.

In accordance with a further embodiment of the present disclosure, the back-end system comprises a near field communication (NFC) antenna for performing wireless data communication with a portable terminal, wherein the portable terminal supplies wireless power to the back-end system by generating an induced current in the NFC antenna.

In accordance with a further embodiment of the present disclosure, the induced current is coupled across the graphene films of the detection region and the control region for determining the surface resistance of the graphene films.

In accordance with a further embodiment of the present disclosure, the back-end system comprises an analog-to-digital converter (ADC) configured to receive analog signals representative of the surface resistance of the graphene films or a difference in the surface resistance of the graphene films, and output digital signals based on the analog signals.

In accordance with a further embodiment of the present disclosure, the ADC is a 14-bit sigma-delta ADC, or the ADC is integrated as a part of a processor of the integrated device.

In accordance with a further embodiment of the present disclosure, the integrated device includes a processor. The back-end system comprises a thermistor or other temperature sensors for detecting a breath temperature of the breath sample. The back-end system is configured to monitor a variation in the surface resistance of the graphene film of the detection region for determining a respiratory rate. The processor is configured to process the digital signals, the breath temperature, and the respiratory rate, and transmit to an external controller for presenting in a user interface.

In accordance with a further embodiment of the present disclosure, the external controller is configured to process the digital signals, the breath temperature, and the respiratory rate; execute an integrated medical evaluation on the user; and determine an action that the user needs to take.

In accordance with a further embodiment of the present disclosure, the integrated medical evaluation is configured to help the user or medical practitioners to understand conditions of the user and whether the user is inflected with COVID 19, identify a high-grade fever, hyperpyrexia, and breath difficulties, and determine whether an immediate medical attention is needed.

In accordance with a further embodiment of the present disclosure, the integrated device is embedded and fixed in an underside of a face mask.

In accordance with a further embodiment of the present disclosure, the integrated device is built into a breathalyzer or other blowing devices. The breathalyzer includes an inlet for performing rapid testing of an individual by exhaling to the inlet.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects and advantages of the present invention are disclosed as illustrated by the embodiments hereinafter.

### BRIEF DESCRIPTION OF THE DRAWINGS

The appended drawings contain figures to further illustrate and clarify the above and other aspects, advantages, and features of the present disclosure. It will be appreciated that these drawings depict only certain embodiments of the present disclosure and are not intended to limit its scope. It will also be appreciated that these drawings are illustrated for simplicity and clarity and have not necessarily been depicted to scale. The present disclosure will now be described and explained with additional specificity and detail through the use of the accompanying drawings in which:

FIG. 1 depicts a conceptual illustration SARS-CoV-2 infection and transmission in the manner of aerosol;

FIG. 2 depicts application scenarios of the integrated device of the present disclosure;

FIG. 3 depicts the capture mechanism of aerosol by aqueous solution relying on gas-liquid interface;

FIG. 4 depicts the integrated device for fast diagnosis of COVID-19, in accordance with certain embodiments of the present disclosure;

FIG. 5 depicts an exploded view of the integrated device, in accordance with certain embodiments of the present disclosure;

FIG. 6 depicts a system block diagram of the integrated device, in accordance with certain embodiments of the present disclosure;

FIG. 7A depicts the internal structure of the microfluidic channel, in accordance with certain embodiments of the present disclosure;

FIG. 7B depicts the external structure of the microfluidic channel, in accordance with certain embodiments of the present disclosure;

FIG. 8 depicts the capture mechanism of SARS-CoV-2 by antibody to modify on a graphene film, in accordance with certain embodiments of the present disclosure;

FIG. 9 depicts the integrated device of FIG. 4 integrated into a built-in mask, in accordance with certain embodiments of the present disclosure;

FIG. 10 depicts a conceptual illustration of a user with the mask of FIG. 9 walking through the NFC door for biosafety checking;

FIG. 11 shows the detection performance of the integrated device to spike protein of SARS-CoV-2;

FIG. 12 shows the relationship between the current and the logarithm of the spike protein concentration of SARS-CoV-2;

FIG. 13 shows the 10-minute current waveform of the integrated device to MERS-CoV, SARS-CoV, and SARS-CoV-2;

FIG. 14 shows the recognition of the MERS-CoV, SARS-CoV, and SARS-CoV-2 using the integrated device;

FIGS. 15A-15D show the detection performance of the integrated device to four types of prevalent strains, including WT (FIG. 15A), Alpha (FIG. 15B), Delta (FIG. 15C), and Omicron (FIG. 15D).

FIG. 16 shows the use of CAT #40592-MM45 in the integrated device for recognizing Delta variant of the SARS-CoV-2;

FIG. 17 shows the use of CAT #40591-MM48 in the integrated device for recognizing Omicron of the SARS-CoV-2;

FIGS. 18A-18D show the voltage response curves of four types virus variants with concentrations from 500 fg ml⁻¹ to 1 ng ml⁻¹.

FIG. 19 shows a typical temperature curve of a breath sample;

FIG. 20 shows a comparison between the breath sample and the forehead temperature obtained from ten subject individuals;

FIG. 21 shows an exemplary waveform on the changes in current for a series of rapid breath with a fast response rate;

FIG. 22 is a zoomed view of FIG. 21 showing one breathing cycle;

FIG. 23 shows the stability of the current measurement in wind, walk and run situations;

FIG. 24 shows the breath characteristics of three breath mode: rapid breath, normal breath, and heavy breath; and

FIG. 25 shows the universality of the integrated device for monitoring different subject individuals.

### DETAILED DESCRIPTION OF THE INVENTION

The following detailed description is merely exemplary in nature and is not intended to limit the disclosure or its application and/or uses. It should be appreciated that a vast number of variations exist. The detailed description will enable those of ordinary skilled in the art to implement an exemplary embodiment of the present disclosure without undue experimentation, and it is understood that various changes or modifications may be made in the function and structure described in the exemplary embodiment without departing from the scope of the present disclosure as set forth in the appended claims.

The benefits, advantages, solutions to problems, and any element(s) that may cause any benefit, advantage, or solution to occur or become more pronounced are not to be construed as a critical, required, or essential features or elements of any or all of the claims. The invention is defined solely by the appended claims including any amendments made during the pendency of this application and all equivalents of those claims as issued.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The terms “comprising,” “having,” and “including” or any other variation thereof, are to be construed as open-ended terms (i.e., meaning “including, but not limited to,”) unless otherwise noted. The use of any and all examples, or exemplary language (e.g., “such as”) provided herein, is intended merely to illuminate the invention better and does not pose a limitation on the scope of the invention unless otherwise claimed. No language in the specification should be construed as indicating any non-claimed element as essential to the practice of the invention. Further, unless expressly stated to the contrary, “or” refers to an inclusive “or” and not to an exclusive “or”. For example, a condition A or B is satisfied by any one of the following: A is true and B is false, A is false and B is true, and both A and B are true. Terms of approximation, such as “about”, “generally”, “approximately”, and “substantially” include values within ten percent greater or less than the stated value.

Unless otherwise defined, all terms (including technical and scientific terms) used in the embodiments of the present invention have the same meaning as commonly understood by an ordinary skilled person in the art to which the present invention belongs.

The described device can have any suitable components or characteristics that allow the device to perform fast self-diagnosis of COVID-19. In one preferred embodiment, the device is integrated into a face mask. In other alternative embodiments, the device can have any suitable designs that allow the device to be integrated into a gas mark, an oxygen mask, or a ventilator. One having ordinary skill in the art would understand that the current disclosure is also applicable to a breathalyzer and other personalized and wearable devices, such as, but without limiting, a patch, or a wristband.

Coronaviruses can cause upper respiratory tract disease in humans. In certain serious situations, the patient may suffer from difficulty breathing or shortness of breath, which could be fatal. SARS-CoV-2 is a kind of betacoronavirus, it has a single-positive strand RNA genome. The severe acute respiratory syndrome coronavirus (SARS-CoV) and the Middle East respiratory syndrome coronavirus (MERS-CoV) are two others betacoronaviruses that can cause serious epidemics. Compared with MERS-CoV, SARS-CoV-2 is more similar to SARS-CoV, according to the phylogenetic analysis. The described integrated device is intended to recognize SARS-CoV-2 and its variants (e.g., Alpha, Delta, and Omicron). In other embodiments, the integrated device can specifically recognize different viral antigens by using different antibodies. Therefore, the description of recognizing SARS-CoV-2 for the detection of COVID-19 is merely exemplary in nature and is not intended to limit to such application. In fact, the integrated device may also be modified to detect other viruses associated with other diseases, such as SARS-CoV and MERS-CoV.

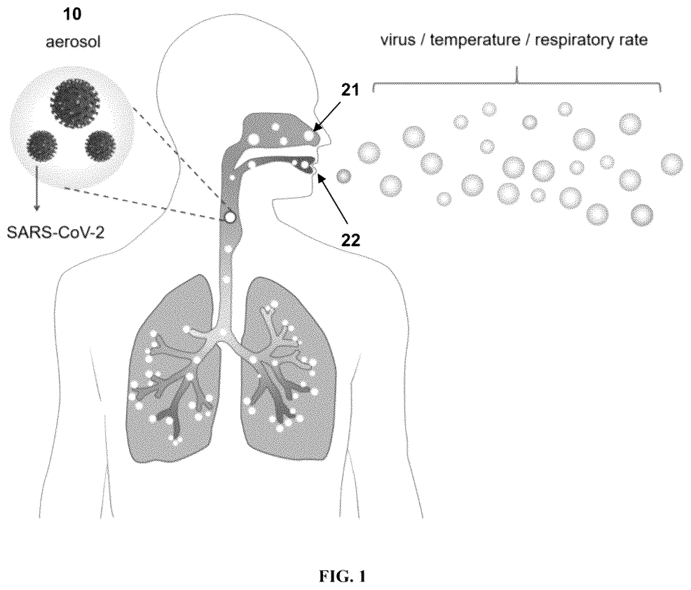

With reference to FIG. 1, the infection and transmission of SARS-CoV-2 are conceptually depicted. The principle of aerosol 10 generations and transmission is made from the lung of an infected patient, through the nasal cavity 21 and the oral cavity 22, to the external environment. The size of the aerosol 10 is generally around 5 μm-100 μm. The aerosol 10 can be a liquid droplet or a suspension of solid particles, which can be generated from breathing, sneezing, coughing, and talking. These SARS-CoV-2 viruses can be attached to the aerosol 10 and spread into the environment. The aerosol 10 is very effective in transmission, and may travel a long distance to spread the virus.

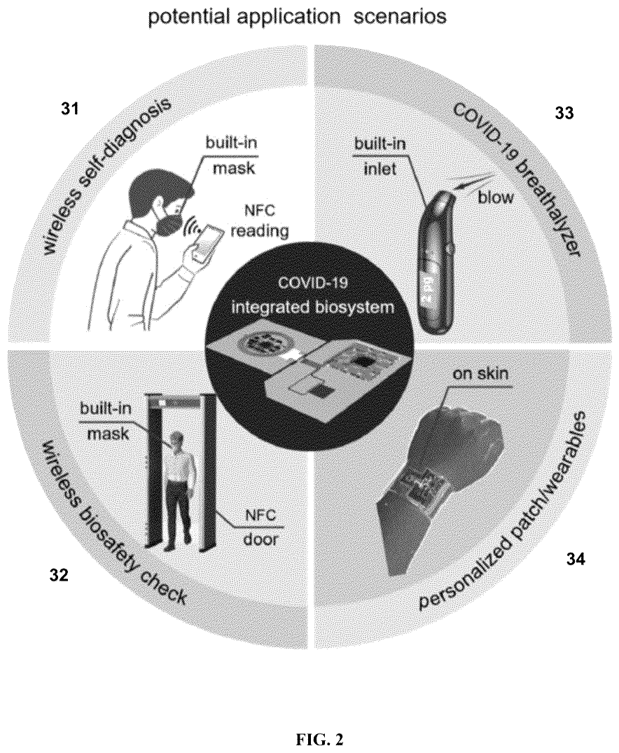

Inspired by the need for a flexible and multifunctional system for fast diagnosis of COVID-19, the present disclosure provides a novel integrated device that can recognize SARS-CoV-2 and other variants. Information that can reflect the health status of the user is collected, including the laden virus, the breath temperature, and the respiratory rate. The purpose of the integrated device is to capture virus-laden aerosol using a gas-liquid interface in a chamber and a plurality of gas channels. Preferred application scenarios of the integrated device are exemplarily illustrated in FIG. 2. The integrated device can be integrated into a mask as a “built-in mask” for performing self-diagnosis 31. The user can activate the diagnosis using the near field communication (NFC) reader of a smartphone. Similarly, the integrated device can be used for performing biosafety check 32 using the “built-in mask”. The check is performed when the user walks through an NFC device, such as an NFC door or an NFC gate. The NFC device is configured to communicate with the built-in mask to identify whether the person walking through is inflected. The third application is a COVID-19 breathalyzer 33, which is a standalone device used for testing the presence of the virus in the breath. Lastly, the integrated device can also achieve personalized customization for users as a patch or a wristband 34. The patch may also be integrated into different wearables, garments, or other articles.

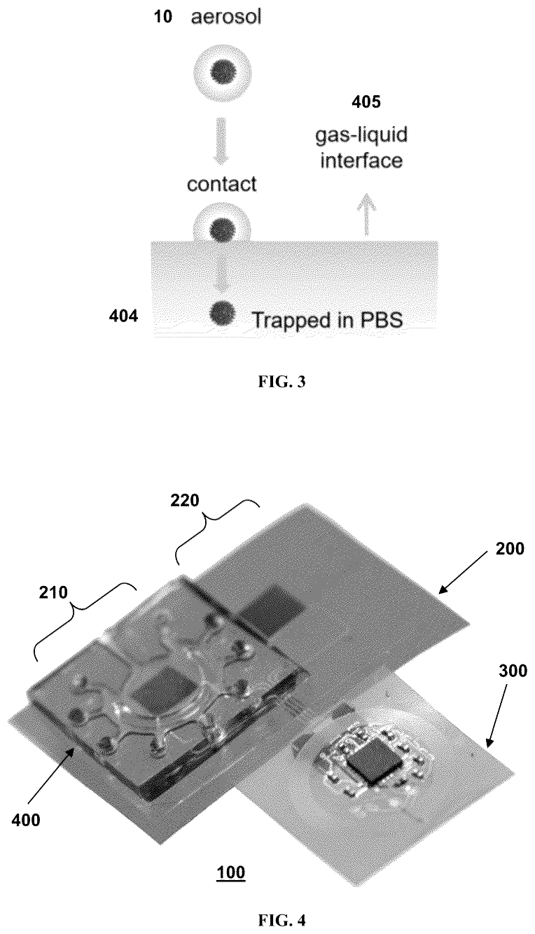

FIG. 3 shows the capture mechanism of aerosol by an aqueous solution relying on a gas-liquid interface 405. The concept behind the integrated device is to allow the virus-laden aerosol 10 to enter into a chamber, which is in contact with the liquid and is trapped in the pre-injected Phosphate-buffered saline (PBS) 404 or other pre-injected solutions.

The present disclosure relates to an integrated device for analyzing a breath sample, which is generally designated as 100. More specifically, but without limitation, the present disclosure provides a flexible, capable of communicating wirelessly, and battery-less multifunctional device for analyzing a breath sample. For realizing the preferred application scenarios, the integrated device 100 is arranged to be placed close to the nasal cavity 21 or the oral cavity 22 when use, so that more breath samples can be projected into the integrated device 100 for the detection of a selected virus. As shown in FIG. 4, the integrated device 100 comprises a front-end system 200, a back-end system 300, and a microfluidic channel 400. Preferably, the integrated device 100 is made of a flexible material so as to be deformed to fit the shape of the face mask or other viral antigen detection device. The microfluidic channel 400 is configured to receive the breath sample. The front-end system 200 comprises a detection region 210 and a control region 220. The microfluidic channel 400 is arranged to cover the detection region 210 so that the breath sample is analyzed by the detection region 210, while the control region 220 is configured to provide reference data for comparing with that from the detection region 210.

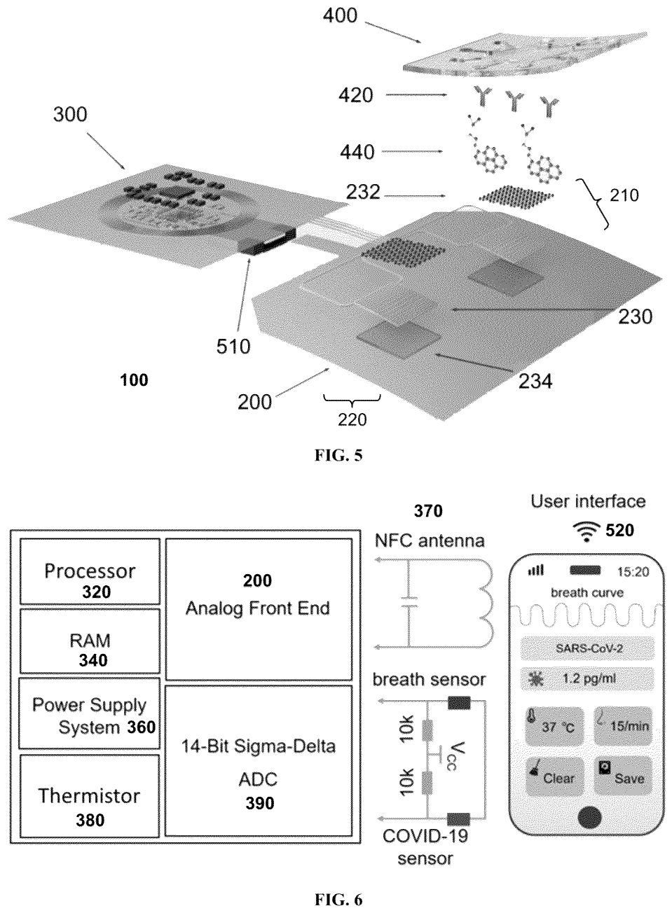

Turning to the exploded view in FIG. 5, on the front-end system 200, the detection region 210 and the control region 220 have similar structures, each comprising an interdigital electrode 230, a graphene film 232, and optionally an acrylic sheet 234. In certain embodiments, the interdigital electrode 230 is covered by the graphene film 232, and adhesively attached to the acrylic sheet 234. The detection region 210 is covered by the microfluidic channel 400 from above so as to allow the interdigital electrode 230 of the detection region 210 to be exposed to the breath sample and to capture the selected virus. In contrast, the control region 220 is not covered the microfluidic channel 400 and so the interdigital electrode 230 of the control region 220 cannot capture the selected virus from the breath sample. In one example, the graphene film 232 comprises a single-layer of graphene. It is apparent that the graphene film 232 may otherwise comprise double-layer graphene, triple-layer graphene, or multi-layer graphene, without departing from the scope and spirit of the present disclosure. Optionally, an acrylic sheet 234 may be used for providing support to the detection region 210 and the control region 220.

The microfluidic channel 400 is configured to hold the pre-injected PBS 404 for defining a gas-liquid interface 405. The pre-injected PBS 404 is injected with a molecular linker 440 and a spike-binding antibody 420 for capturing the spike protein of the selected virus, such as SARS-CoV-2 and other variants. Different spike-binding antibodies 420 shall be used to capture different viruses. In one example, the spike-binding antibody 420 may include an anti-Coronavirus spike neutralizing antibody (40592-MM45) for recognizing a Delta variant, or an anti-Coronavirus spike neutralizing antibody (40591-MM48) for recognizing an Omicron variant, or both. In certain embodiments, the spike-binding antibody 420 may be other SARS-CoV-2-Spike-binding antibodies that bind other viral antigens. In certain embodiments, the spike-binding antibody 420 is not limited to one type of antibody. The molecular linker 440 is a molecular chain of various lengths for forming a non-covalent conjugation connecting the spike-binding antibody 420 to the graphene film 232, which can enhance the coupling efficiency through Amino (—NH2) and carboxyl (—COOH) groups. In one embodiment, the molecular linker 440 comprises cross-linking collagen polymers using 1-ethyl-3-(3-dimethylaminopropyl)carbodiimide (EDC) and N-hydroxysuccinimide (NHS). Particularly, the first end of the EDC/NHS (carboxyl) combines with the graphene film 232, and the second end of the EDC/NHS (Amino) combines with the spike-binding antibody 420. It is apparent that the molecular linker 440 used in the manufacture of the microfluidic channel 400 of the present disclosure may be other reagents that can enhance the coupling efficiency of the spike-binding antibody 420 and the graphene film 232 without departing from the scope and spirit of the present disclosure. Once the spike protein of the selected virus is tightly captured by the spike-binding antibody 420, the molecular linker 440 can link the selected virus to the graphene film 232 of the detection region 210. As the graphene films 232 of the detection region 210 and the control region 220 shall have similar surface resistance, if the selected virus is captured by the spike-binding antibody 420 and linked to the graphene film 232 of the detection region 210, the surface resistance of graphene will be increased. Therefore, the graphene film 232 of the detection region 210 is found to have a surface resistance higher than that of the graphene film 232 of the control region 220 when the detection region 210 is exposed to the selected virus. The difference in surface resistance may also be used to monitor the respiratory rate. When the aerosol in expiration skips over the graphene surface 232, the surface resistance increases. When inhaling air, the air absorbs aerosol from the graphene surface to evaporate the aerosol, and the surface resistance is decreased. The variation in the surface resistance of the graphene film 232 can be monitored by the back-end system 300 for determining the respiratory rate.

The control region 220 has another interdigital electrode 230 and another graphene film 232 with essentially the same size and shape as that in the detection region 210 but without being covered by the microfluidic channel 400. The control region 220 and the detection region 210 may be placed next to each other. Molecular linker 440 and spike-binding antibody 420 are not present in the control region 220, and so the graphene film 232 of the control region 220 is not modified by the selected virus.

The front-end system 200 and the back-end system 300 may be operably connected with each other via one or more communication links, which may be wired (e.g. cable buses) or wireless. In the illustrated embodiment, the back-end system 300 may include one or more connectors 510 for the realization of the wired communication links. The back-end system 300 is configured to detect and compare the surface resistance of the graphene films 232 of the detection region 210 and the control region 220 for determining whether the selected virus is present in the breath sample. If the surface resistance of the graphene film 232 of the detection region 210 is increased, it is expected that the spike-binding antibody 420 tightly captures the spike protein of the selected virus. Therefore, the back-end system 300 will determine that the selected virus is present in the breath sample.

FIG. 6 provides the system block diagram of the integrated device 100. In certain embodiments, the back-end system 300 comprises a processor 320, a random access memory (RAM) 340, a power supply system 360, a thermistor 380, an analog-to-digital converter (ADC) 390, and an NFC antenna 370. The back-end system 300 is communicable with an external controller, such as a smartphone 520 or a cloud processor, using NFC so that the acquired data can be analyzed and presented to the user.

The NFC antenna 370 is arranged to perform wireless data communication with a portable terminal. The portable terminal supplies wireless power to the back-end system 300 by generating an induced current in the coil of the NFC antenna 370. In certain embodiments, the NFC antenna 370 generates a constant voltage of 0.2 mV, which operates as the power supply system 360 to power the ADC 390, the processor 320, the NFC antenna 370, and the thermistor 380. The power generated from the NFC antenna 370 is also used to sense the surface resistance of the graphene films 232. In certain embodiments, the induced current is coupled across the graphene films 232 of the detection region 210 and the control region 220 for determining the surface resistance of the graphene films 232. When the selected virus is present in the breath sample, the surface resistance of the graphene film 232 of the detection region 210 is increased, so the current across the graphene film 232 is decreased. On the other hand, the surface resistance of the graphene film 232 of the control region 220 is not changed, so the current is higher than that in the detection region 210.

The ADC 390 is configured to receive analog signals representative of the surface resistance of the graphene films 232, or analog signals representative of the difference in the surface resistance of the graphene films 232 of the detection region 210 and the control region 220. Then, the ADC converter 390 outputs digital signals based on the analog signals. The ADC 390 may be a sigma-delta ADC. In one example, the ADC 390 is a 14-bit sigma-delta ADC. Alternatively, the ADC 390 may be integrated as a part of the processor 320, so that the analog signals representative of the surface resistance are coupled directly to the processor 320 for determining the difference in the surface resistance of the graphene films 232 of the detection region 210 and the control region 220.

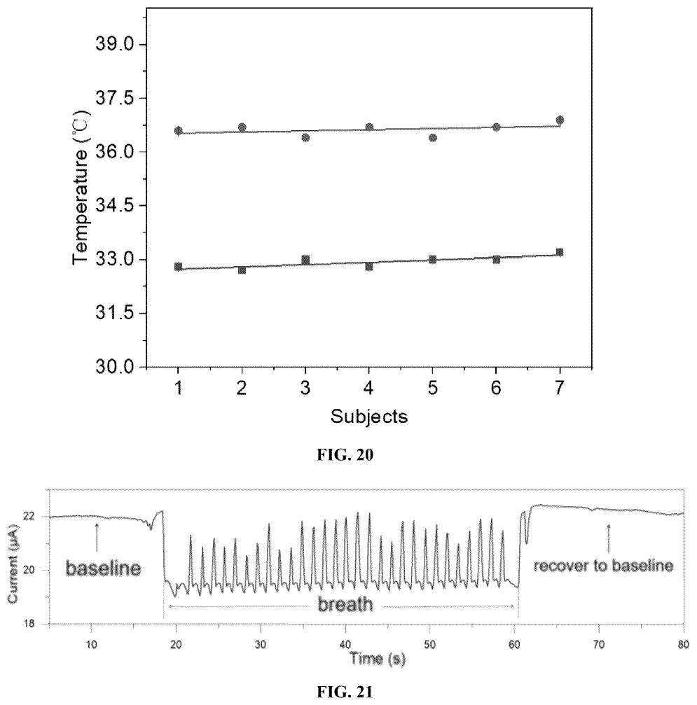

The thermistor 380, or other temperature sensors, is provided for detecting the breath temperature of the breath sample. FIG. 19 shows a typical temperature curve of the breath sample when the room temperature is about 18° C. and the breath temperature is about 32.5° C. As the temperature is significantly increased, the breath duration and the respiratory rate can also be determined. The breath temperature is mapped to a temperature compensation curve for estimating a forehead temperature. The forehead temperature is used as a reference to evaluate whether there is a body fever. To estimate the forehead temperature, the present disclosure provides a comparison between the breath temperature and the forehead temperature, as obtained from ten subject individuals, as shown in FIG. 20. The compensation value is about 3.7° C., which can be used as a reference for evaluating the forehead temperature of the user. This data is used to form part of a temperature compensation curve. It is apparent that the compensation value may vary depending on the environmental conditions and the individual conditions. The temperature difference is consistent, and the estimation of the forehead temperature can rely on the breath temperature. The compensation value may vary for infants and elderlies. A separate temperature compensation curve may be applied to improve the accuracy. The temperature compensation curve may also be adapted to each individual user, for example, by a machine learning algorithm. In certain embodiments, the temperature compensation curve is stored in the RAM 340, which may be updated intermittently via the NFC antenna 370. Alternatively, the temperature compensation curve may be stored in the external controller. When the breath temperature is received by the external controller, the breath temperature is processed to determine the forehead temperature.

The processor 320 and the RAM 340 are configured to process and store the digital signals obtained from the ADC 390, and transmit the digital signals to a smartphone or a cloud processor via the NFC antenna 370. In certain embodiments, the processor 320 is further configured to process the breath temperature obtained from the thermistor 380 or other temperature sensors, and the respiratory rate, and transmit the breath temperature and the respiratory rate to the external controller, such as the smartphone 520 or the cloud processor. In certain embodiments, an external controller is configured to process the digital signals representative of the surface resistance of the graphene films 232, the breath temperature, and the respiratory rate, execute an integrated medical evaluation on the user, and determine an action that the user needs to take. The integrated medical evaluation is configured to help the user or the medical practitioners to understand the conditions of the user and whether the user is inflected with COVID 19. In one example, the integrated medical evaluation may identify a high-grade fever, hyperpyrexia, and breathing difficulties, which may be considered as an emergency. The user or the medical practitioners should be promptly alerted such that immediate medical attention is provided. The obtained data may be presented in a mobile application or a website, which is collectively referred to as a user interface.

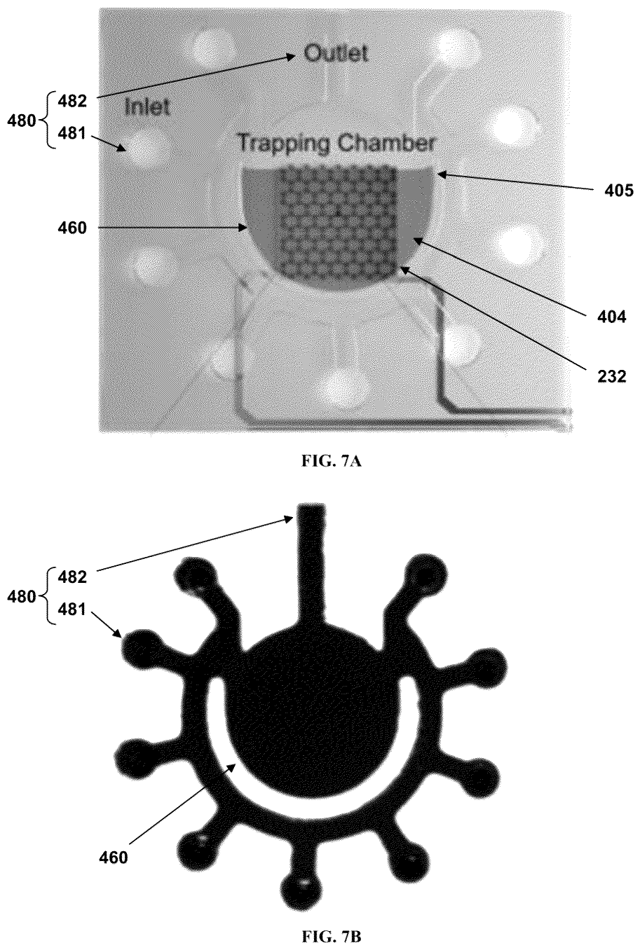

FIGS. 7A-7B show the structure of the microfluidic channel 400. The microfluidic channel 400 is placed above the detection region 210, which enables the detection region 210 to detect the presence of the selected virus in the breath sample. The microfluidic channel comprises a plurality of gas channels 480 and a trapping chamber 460 holding pre-injected PBS 404 or other pre-injected solutions for defining a gas-liquid interface 405. In certain embodiments, the trapping chamber 460 may have shapes other than the illustrated circular shape. The plurality of gas channels 480 comprise plural gas inlets 481 and a gas outlet 482. In one example, the plurality of gas channels 480 consist of 9 gas inlets 481 and one gas outlet 482. The plurality of gas channels 480 are arranged outside and around the trapping chamber 460. In particular, the gas outlet 482 is positioned proximate to a gas side of the gas-liquid interface 405, while the plural gas inlets 481 are positioned proximate to a liquid side of the gas-liquid interface 405. Therefore, the breath sample is arranged to enter the trapping chamber 460 via the plural gas inlets 481 and passes through the gas-liquid interface 405 to the gas outlet 482. The purpose of this arrangement is to trap the aerosol in the breath sample in the pre-injected PBS 404 or other pre-injected solutions. In certain embodiments, the pre-injected PBS 404 may comprise more than one type of spike-binding antibody 420 for identifying different variants of the selected virus. The selected virus, if being carried by the aerosol of the breath sample, is collected and trapped in the pre-injected PBS 404 for performing the virus detection. As the microfluidic channel 400 is placed above the detection region 210, the graphene film 232 and the interdigital electrode 230 of the detection region 210 are placed inside the trapping chamber 460 and are immersed in the pre-injected PBS 404. The selected virus will not directly cause any changes to the surface resistance of the graphene film 232. Therefore, it is necessary to use a spike-binding antibody 420 to capture the spike protein of SARS-CoV-2 and its variants (e.g., Alpha, Delta, and Omicron), and link the captured SARS-CoV-2 to the graphene film 232 by a molecular linker 440. The surface resistance of the graphene film 232 of the detection region 210 is increased when exposed to the selected virus.

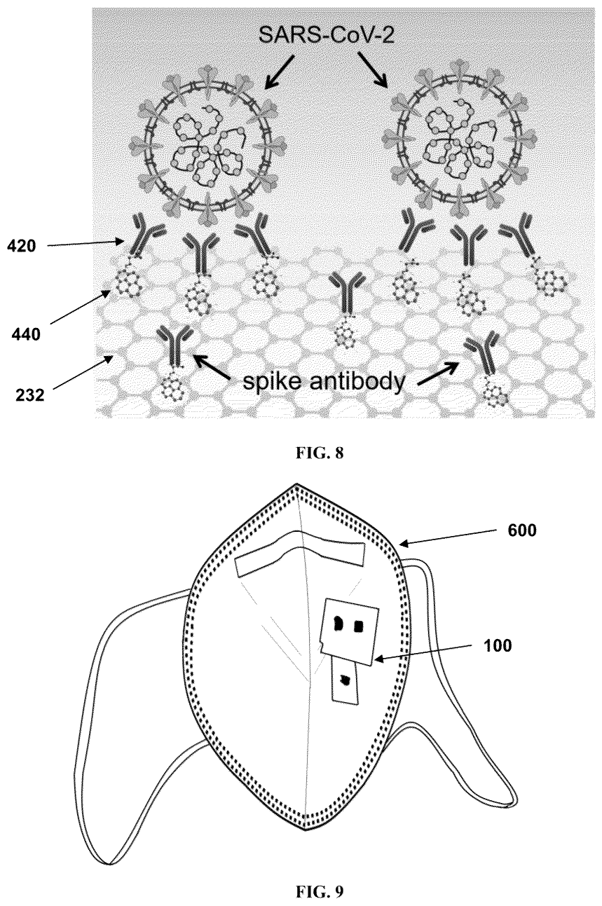

FIG. 8 illustrates the capture mechanism of SARS-CoV-2 by antibody to modify the graphene film 232. After SARS-CoV-2 is dissolved in the pre-injected PBS 404, the spike-binding antibody 420 will capture the Spike S1 protein of SARS-CoV-2, which changes the surface resistance of graphene film 232.

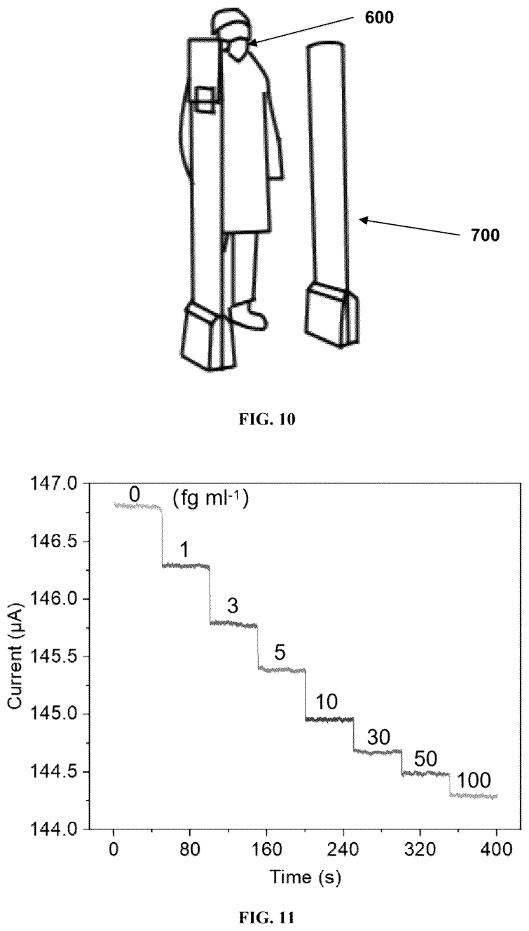

The integrated device 100 can be implemented in various applications. As illustrated in FIG. 9, the integrated device 100 is embedded and fixed in the underside of a face mask 600 facing the user. In one example, the face mask 600 is a KN95 mask. After embedding in the face mask 600, the integrated device 100 can be used to achieve wireless self-diagnosis of the user using a smartphone. Additionally, there are many large consuming places (e.g., supermarkets, shopping malls, scenic areas) and security places (e.g., customs, hospitals, airports, schools, railway stations) equipped with NFC doors 700, which help to achieve wireless biosafety check. This is conceptually illustrated in FIG. 10. By wearing a face mask 600 with the integrated device 100, when the user stands outside the NFC door 700, no response is generated. When the user walks through the NFC door 700, the infection information is read by the NFC door 700 and recognized. Then an alert, such as a red light, is activated to remind the safety personnel. After the user passes through the NFC door 700, the alert is turned off. Similarly, when a healthy person wearing the face mask 600, the NFC door 700 shows a green light and reminds that the user is healthy. The green light is turned off after the user passes through the NFC door 700.

Additionally, except using in the face mask 600, the integrated device 100 can also be built into a breathalyzer or other blowing devices, which allows rapid testing of an individual by exhaling to an inlet of the breathalyzer. The user can blow into the inlets to perform the biosafety test. If the user is inflected, the virus-laden aerosol will be collected and detected by the built-in integrated device 100. In other embodiments, this integrated device 100 may be customized and integrated into other wearables, such as a wristband, without departing from the scope and spirit of the present disclosure.

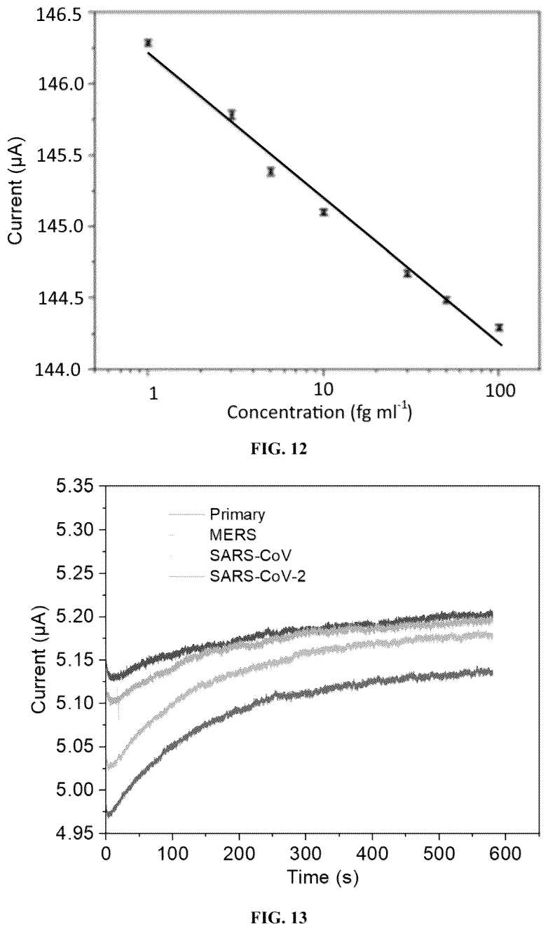

To verify the recognition performance of integrated device 100, pseudovirus of different concentrations (1 fg/ml to 100 fg/ml) of the Spike 51 protein of SARS-CoV-2 are added to a graphene film 232. The current across the graphene film 232 is measured for each sample when a constant voltage of 0.2V is applied. The result is shown in the graph of FIGS. 11 and 12. When the spike-binding antibody 420 captures the Spike S1 protein of SARS-CoV-2, the surface resistance of graphene film 232 increases, and the corresponding current decreases. When the concentration of the Spike S1 protein of SARS-CoV-2 continues to increase, the corresponding current decreases further.

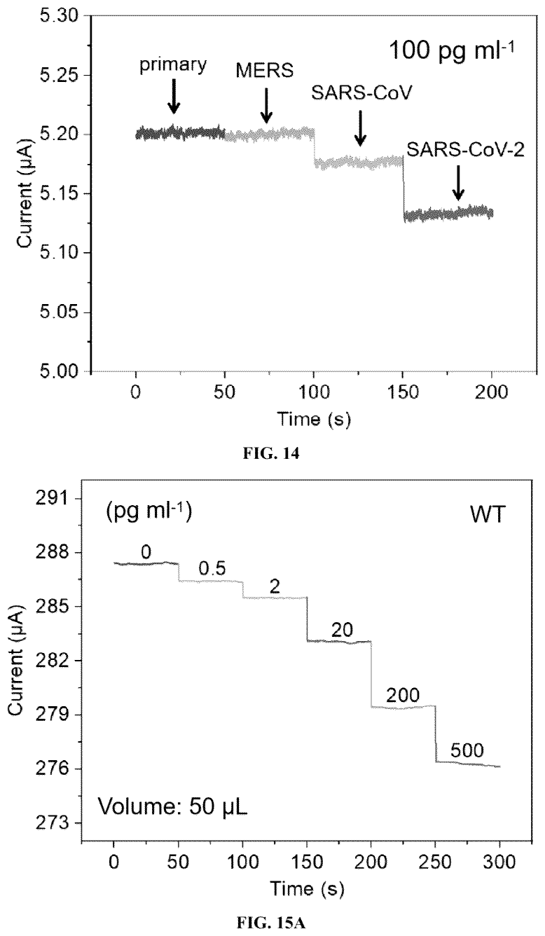

To verify the specificity of the antibody to SARS-CoV-2, pseudovirus with spike protein of MERS-CoV, SARS-CoV, and SARS-CoV-2 (50 μL, 100 pg ml⁻¹) are added on graphene film 232 in sequence. As shown in FIGS. 13 and 14, a blank curve (primary) is run for 10 minutes until it nears stable. The stable current of MERS-CoV is close to overlapping with the blank curve. No significant difference was formed between them. In striking contrast, a distinguishable current difference formed between SARS-CoV and the blank curve. And a marked difference formed between SARS-CoV-2 and the blank curve. These response results agree with the information provided by the modified antibody (Cat: 40150-R007) of SB SinoBiological. SARS-CoV-2 has four structural proteins: spike (S), envelope (E), matrix (M), and nucleocapsid (N). These structural proteins share 76%-95% sequence identity with those of SARS-CoV. This percentage homology is reduced to 30%-40% for MERS-CoV. Similarly, the nucleocapsid protein of SARS-CoV-2 is 90% identical to that of SARS-CoV. The distinguishable current difference between SARS-CoV and the blank curve is expected. However, the lack of specific selectivity in this particular case is not a major concern because no SARS-CoV cases were reported up to now. Therefore, it can be reasonably assumed that this particular case will not affect the specific recognition of COVID-19 cases in practical usage.

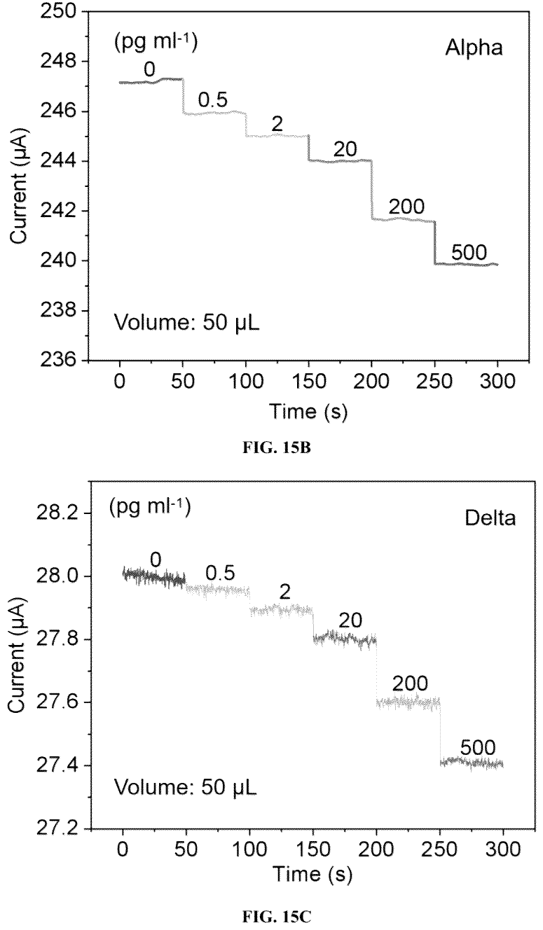

Turning to FIGS. 15A-15D, the four graphs show the detection performance of the integrated device 100 for four typical variants of the SARS-CoV-2, including wild type (WT), Alpha, Delta, and Omicron. The testing concentration range is from 0.5 pg ml⁻¹ to 500 pg ml⁻¹. As shown in FIGS. 15A and 15B, the integrated device 100 show recognizable responses to WT and Alpha at a minimum detection concentration of 0.5 pg ml⁻¹. The current across the graphene film 232 decreases when the concentration increases. As the mutation degree increases, the minimum detection concentration slightly increases. For example, the response current for the Delta variant at 0.5 pg ml⁻¹ is almost as low as that for 0 pg ml⁻¹, as shown in FIG. 5C. The Delta variant is only recognizable at 2 pg ml⁻¹. This recognizable minimum detection concentration of Omicron is further increased to 5 pg ml⁻¹ due to a greater mutation degree (FIG. 5D).

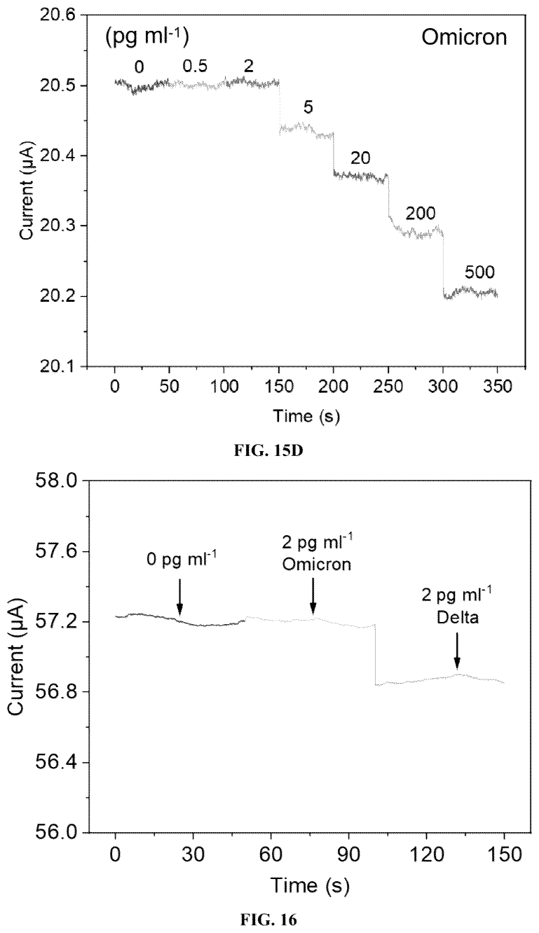

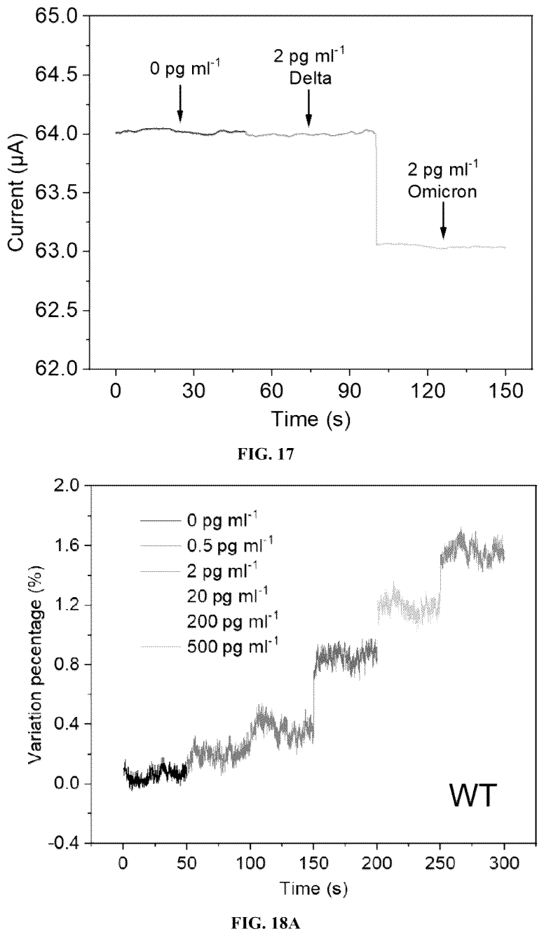

In addition to the ability to respond to WT, Alpha, Delta, and Omicron, by changing the spike-binding antibody 420 on the graphene film 232, the integrated device 100 can especially differentiate the epidemic strains, for example, Delta and Omicron. For example, the spike-binding antibody 420 may be CAT #40592-MM45 for recognizing Delta (not recognizing Omicron), and may be CAT #40591-MM48 for recognizing Omicron (not recognizing Delta). As shown in FIGS. 16 and 17, after changing the spike-binding antibody 420 to CAT #40592-MM45, the integrated device 100 shows specific recognition to Delta. Similarly, after changing the spike-binding antibody 420 to CAT #40591-MM48, the integrated device 100 shows specific recognition to Omicron. This function means that the users may use the integrated device 100 to determine whether they are infected with the SARS-CoV-2, and also know the type of the variant.

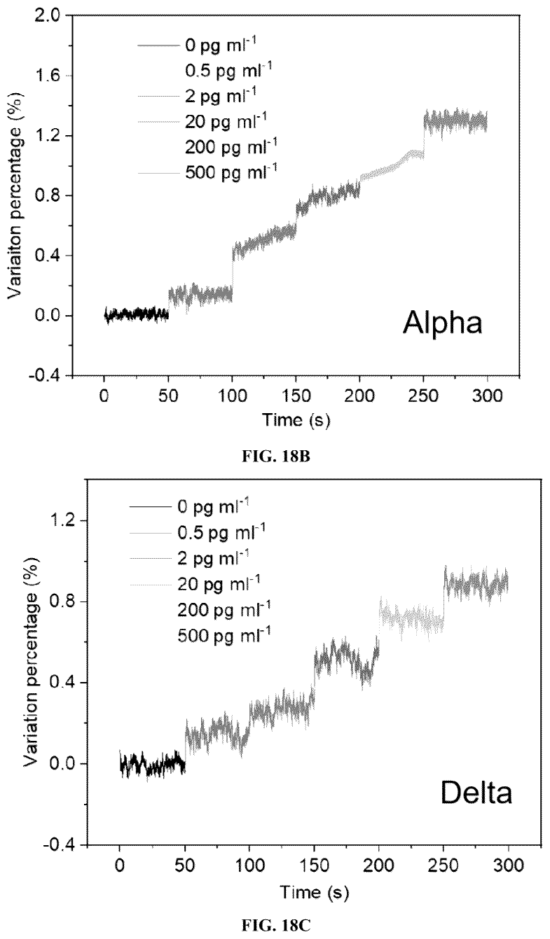

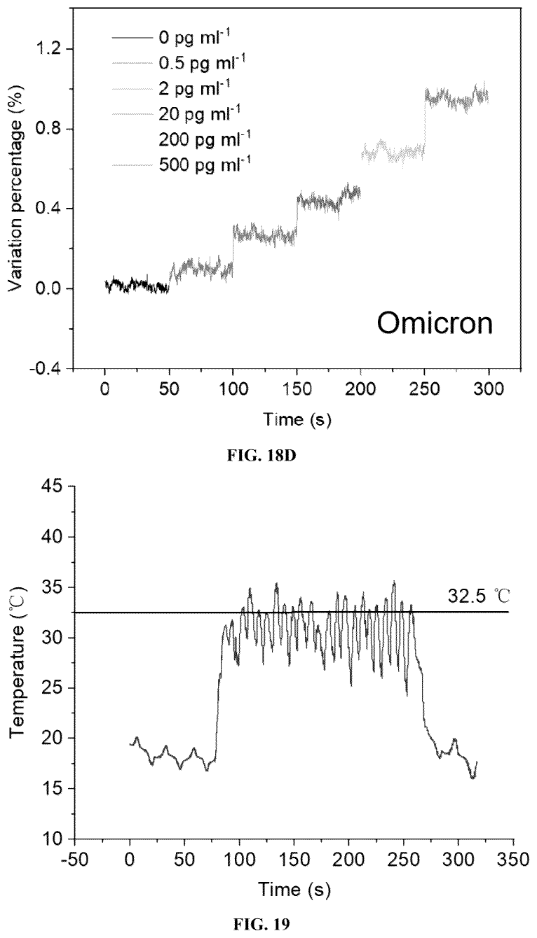

Apart from the use of pseudovirus, various variants of the SARS-CoV-2 virus, including WT, Alpha, Delta, and Omicron, are tested on the integrated device 100 in a physical containment level 3 laboratory. The integrated device 100 shows good detection ability to these four SARS-CoV-2 viruses. The data for the four variants are shown in FIGS. 18A-18D. The virus (50 μL) is added to the graphene film 232 with a constant current of 100 μA applied across the graphene film 232. When the virus was added to the graphene film 232, the spike-binding antibody 420 captures the virus on the graphene film 232, which increases the surface resistance of the graphene film 232. Followed by the increase of the output voltage across the interdigital electrode 230, the integrated device 100 shows responses to all four viruses. The output voltage increases when the concentration increases.

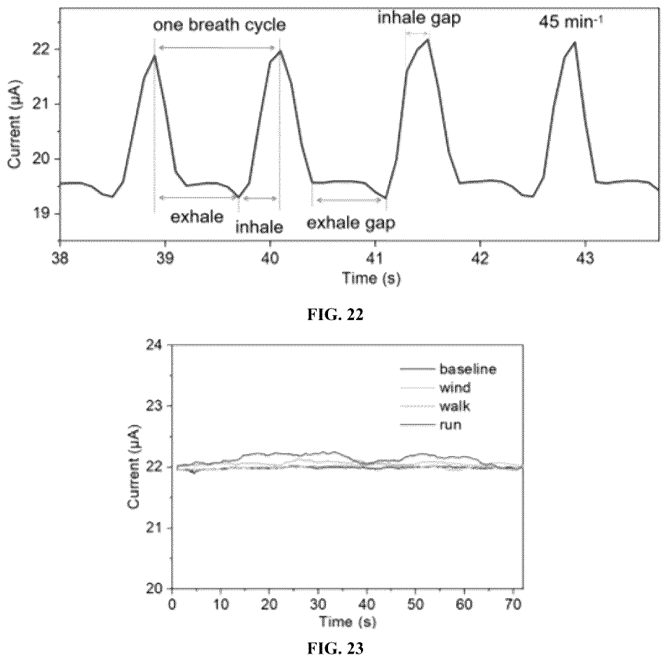

The interdigital electrode 230 covered by the graphene film 232 in the control region 220 can be used to monitor the breath characteristics when a user is wearing the face mask 600. When the exhaled aerosol contacts with the graphene film 232, micro water droplets in the aerosol are absorbed by the graphene film 232, which increases the surface resistance of the graphene film 232. By applying a constant voltage across the interdigital electrode 230, the output current is decreased during exhalation. As a result, the variation in the surface resistance of the graphene film 232 of the control region 220 is monitored for determining the respiratory rate. Similarly, when the user inhales dry air, the micro water droplet on the graphene film 232 is evaporated immediately. The surface resistance can be recovered and the corresponding current raises again. FIG. 21 records a rapid breath curve with a breathing frequency of about 45 breaths per minute. When a user wears the face mask 600, the output current (baseline) is stable at approximately 22 μA. When the user starts breathing, the current across the graphene film 232 records an immediate response during the whole breathing cycle. When the user takes off the face mask 600, the current recovers immediately to the baseline with negligible variations. FIG. 22 is a zoomed view of FIG. 21 showing one breathing cycle. In one breathing cycle, the exhale and inhale characteristics of the user can be monitored. The user firstly generates a fast exhale resulting in a rapid decrease in current, then tends to be gentle when near the end of the inhale (exhale gap). When the user inhales dry air, the current immediately increases, then tends to be gentle when near the end of exhaling (inhale gap).

FIG. 23 shows the stability of the integrated device 100 in the current measurement in different scenarios, such as in wind, walking, and running, the current curves are kept stable and show no regular response to external disturbance.

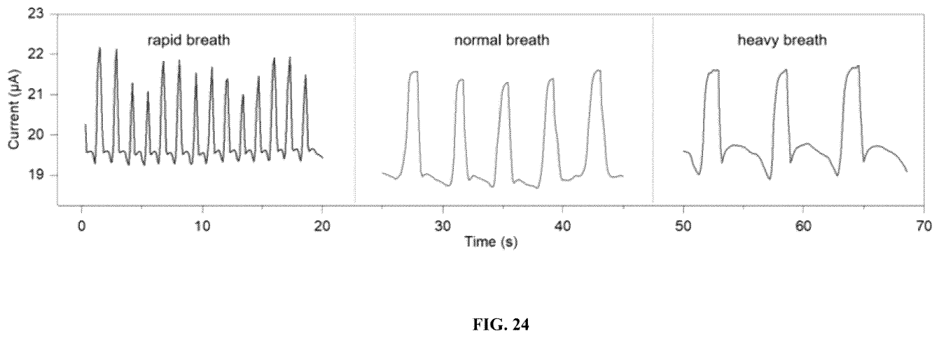

FIG. 24 shows the breath characteristics of three breath modes, for example, rapid breath (45 min⁻¹), normal breath (15 min⁻¹), and heavy breath (10 min⁻¹). The integrated device 100 can monitor the breath characteristics, particularly the respiratory rate, based on the changes in current across the graphene film 232.

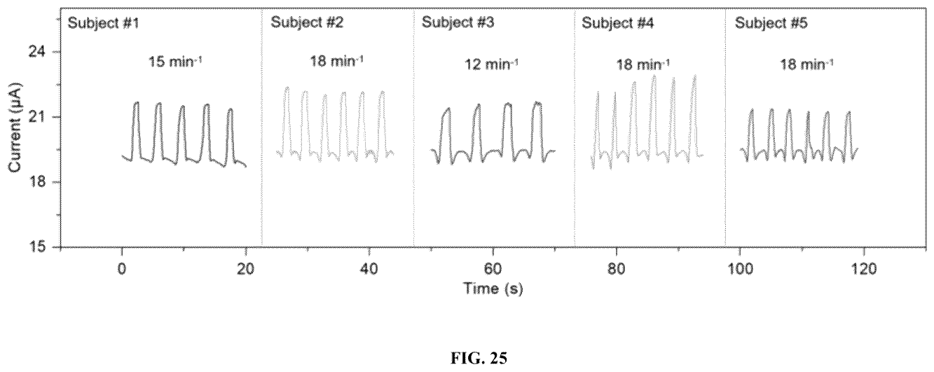

FIG. 25 shows the ability to monitor the breath characteristics of five subject individuals with breath frequencies of 15 min⁻¹, 18 min⁻¹, 12 min⁻¹, 18 min⁻¹, and 18 min⁻¹ respectively. The variation amplitudes of current for the given subject individuals are different due to the diversity of breathing strength and moisture. However, the variation features of the current curve during inhale and exhale are consistent.

This illustrates the fundamental structure of the integrated device for analyzing a breath sample and fast self-diagnosis of COVID-19 in accordance with the present disclosure. It will be apparent that variants of the above-disclosed and other features and functions, or alternatives thereof, may be integrated into many other different applications. The present embodiment is, therefore, to be considered in all respects as illustrative and not restrictive. The scope of the disclosure is indicated by the appended claims rather than by the preceding description, and all changes that come within the meaning and range of equivalency of the claims are therefore intended to be embraced therein.

What is claimed is:

1. An integrated device for analyzing a breath sample, comprising:
a front-end system comprising a detection region and a control region, wherein the detection region and the control region have similar structures, each comprising an interdigital electrode and a graphene film;
a back-end system; and
a microfluidic channel arranged to cover the detection region so as to allow the interdigital electrode of the detection region to be exposed to the breath sample,
wherein:
the graphene film of the detection region has a surface resistance higher than that of the graphene film of the control region when the detection region is exposed to a selected virus; and
the back-end system is configured to detect and compare the surface resistance of the graphene films of the detection region and the control region for determining whether the selected virus is present in the breath sample.

2. The integrated device of claim 1, wherein:
the microfluidic channel comprises a plurality of gas channels and a trapping chamber holding pre-injected Phosphate-buffered saline (PBS) for defining a gas-liquid interface;
the graphene film and the interdigital electrode of the detection region are placed inside the trapping chamber and immersed in the pre-injected PBS; and
the breath sample passes through the gas-liquid interface for capturing the selected virus in the pre-injected PBS.

3. The integrated device of claim 2, wherein the pre-injected PBS is injected with a molecular linker and a spike-binding antibody, wherein the spike-binding antibody is arranged to capture the spike protein of SARS-CoV-2, and the surface resistance of the graphene film of the detection region is increased when exposed to the selected virus with the SARS-CoV-2 linked to the graphene film by the molecular linker.

4. The integrated device of claim 3, wherein the spike-binding antibody is an anti-Coronavirus spike neutralizing antibody (40592-MM45) for recognizing a Delta variant, or an anti-Coronavirus spike neutralizing antibody (40591-MM48) for recognizing an Omicron variant.

5. The integrated device of claim 2, wherein the molecular linker comprises cross-linking collagen polymers using 1-ethyl-3-(3-dimethylaminopropyl)carbodiimide (EDC) and N-hydroxysuccinimide (NHS).

6. The integrated device of claim 5, wherein a first end of the EDC/NHS combines with the graphene film, and a second end of the EDC/NHS combines with the spike-binding antibody.

7. The integrated device of claim 2, wherein the plurality of gas channels comprise plural gas inlets and a gas outlet, which are arranged outside and around the trapping chamber, wherein the gas outlet is positioned proximate to a gas side of the gas-liquid interface, and the plural gas inlets are positioned proximate to a liquid side of the gas-liquid interface.

8. The integrated device of claim 1, wherein the interdigital electrode is covered by the graphene film and adhesively attached to an acrylic sheet.

9. The integrated device of claim 1, wherein the back-end system comprises a thermistor or other temperature sensors for detecting a breath temperature of the breath sample, wherein the breath temperature is mapped to a temperature compensation curve for estimating a forehead temperature.

10. The integrated device of claim 1, wherein the back-end system is further configured to monitor a variation in the surface resistance of the graphene film of the control region for determining a respiratory rate.

11. The integrated device of claim 1, wherein the back-end system comprises a near field communication (NFC) antenna for performing wireless data communication with a portable terminal, wherein the portable terminal supplies wireless power to the back-end system by generating an induced current in the NFC antenna.

12. The integrated device of claim 11, wherein the induced current is coupled across the graphene films of the detection region and the control region for determining the surface resistance of the graphene films.

13. The integrated device of claim 1, wherein the back-end system comprises an analog-to-digital converter (ADC) configured to receive analog signals representative of the surface resistance of the graphene films or a difference in the surface resistance of the graphene films, and output digital signals based on the analog signals.

14. The integrated device of claim 13, wherein the ADC is a 14-bit sigma-delta ADC, or the ADC is integrated as a part of a processor of the integrated device.

15. The integrated device of claim 13 further comprising a processor, wherein:
the back-end system comprises a thermistor or other temperature sensors for detecting a breath temperature of the breath sample;
the back-end system is configured to monitor a variation in the surface resistance of the graphene film of the detection region for determining a respiratory rate; and
the processor is configured to process the digital signals, the breath temperature, and the respiratory rate, and transmit to an external controller for presenting in a user interface.

16. The integrated device of claim 15, wherein the external controller is configured to:
process the digital signals, the breath temperature, and the respiratory rate;
execute an integrated medical evaluation on the user; and
determine an action that the user needs to take.

17. The integrated device of claim 16, wherein the integrated medical evaluation is configured to help the user or medical practitioners to understand conditions of the user and whether the user is inflected with COVID 19, identify a high-grade fever, hyperpyrexia, and breath difficulties, and determine whether an immediate medical attention is needed.

18. The integrated device of claim 1, wherein the integrated device is embedded and fixed in an underside of a face mask.

19. The integrated device of claim 1, wherein the integrated device is built into a breathalyzer or other blowing devices, wherein the breathalyzer comprises an inlet for performing rapid testing of an individual by exhaling to the inlet.
